(19) United States
(12) Patent Application Publication
(10) Pub. No.: US 2020/0199519 A1
(43) Pub. Date: Jun. 25, 2020

(54) SYSTEM AND METHOD FOR CONCENTRATING SUSPENDED SOLIDS PRIOR TO REMOVAL

(71) Applicant: Synata Bio, Inc., Warrenville, IL (US)

(72) Inventor: Robert HICKEY, Okemos, MI (US)

(73) Assignee: Synata Bio, Inc., Warrenville, IL (US)

(21) Appl. No.: 16/633,938

(22) PCT Filed: Jul. 31, 2018
(86) PCT No.: PCT/US2018/044565

§ 371 Date: Jan. 24, 2020

Related U.S. Application Data
(60) Provisional application No. 62/539,238, filed on Jul. 31, 2017.

(51) Int. Cl. C12M 1/00

(52) U.S. Cl. CPC C12M 47/10; C12M 21/12; C12M 29/18; C12M 29/16; C12M 29/04

(57) ABSTRACT

A system and method for concentrating and removing suspended solids from a liquid stream using a filtering device to separate the liquid stream into a permeate stream and a retentate stream, the retentate stream having a higher concentration of particles than the liquid stream or the permeate stream, and providing the retentate stream to a liquid recovery zone to separate the retentate stream into a clarified fluid stream and a concentrated particle stream.

### FIELD OF THE DISCLOSURE

The present disclosure relates to processes and systems for concentrating suspended solids prior to removal from a liquid stream.

### BACKGROUND

Many known process supply microorganism with a feed substrate to biologically convert the substrate into one or more product fuels and/or chemicals. Most known commercial processes suspend the microorganisms in a liquid, typically a fermentation liquid. These biological conversions referred to herein as liquid bioconversion processes produce a significant mass of excess biosolids and other organic matter that requires management. These solids typically comprise dead cell mass and other by-products of the bioconversion.

One type of process involves a liquid bioconversion processes that converts a wide variety of abundant feedstocks, such as natural gas, wood, garbage, industrial gases, gaseous substrates, and other carbon-containing materials, into syngas that is then converted into liquid products such as oxygenated organic compounds which can be fuels and chemicals, in a bioreactor. The process produces a fermentation liquid containing the liquid product and suspended solids comprising organic waste material (bio-waste solids or biosolids). These suspended solids or bio-waste solids or biosolids can be composed of microorganisms, microorganism residue, precipitated proteins and organic by-products. To prevent overconcentration of organic waste material in the fermentation liquid and to recover the liquid product, a liquid stream is removed the fermentor or bioreactor periodically or continually. The liquid stream can comprise fermentation liquid or bioreactor effluent which originates from a bioreactor. The processes have a liquid recovery zone that recovers liquid products from the liquid stream and removes suspended solids from the liquid stream to produce a recycled liquid that returns to the bioreactor substantially free of the suspended solids.

The fermentation liquid could be discarded once any liquid products are removed to prevent an excessive buildup of suspended solids. For most fermentations, discarding fermentation liquid is not viable since this could cause the loss of soluble nutrients and for commercial-scale bioreactors disposal of a large volume of liquid and/or the cost of adding new liquid would prove too costly. A commercial-scale bioreactor may contain over 1 million liters of fermentation liquid. Responsible disposal of the resulting large liquid volumes requires a liquid waste treatment system with a high capacity.

Operating a reactor with a large volume of aqueous broth can be problematic depending upon the capacity of the waste water treatment system. It is likely that the waste water from the bioreactor would have to be slowly discharged to the waste water treatment system to prevent exceeding capacity. Thus, the cost of liquid supply and capital and/or operating cost of liquid treatment usually dictates liquid recovery and reuse by separating bio-waste solids or suspended solids from fermentation liquid. In applications where waste water treatment capacity is limited, waste liquid storage of any excess liquid waste is often employed along with intermittent fermenter shut down when storage capacity reaches its limit and until treatment can again provide sufficient storage. Thus, the downtime of the affected bioreactor would be extended, resulting in a further loss of production. Moreover, the amount of water lost could also be an economic loss.

A particular fermentation that requires liquid recovery from the fermentation effluent is anaerobic fermentations of hydrogen and carbon monoxide to produce oxygenated liquid products such as ethanol, acetic acid, propanol, n-butanol, or other oxygenated organic compounts. The production of these oxygenated organic compounds can require significant amounts of hydrogen and carbon monoxide and fermentation liquid.

For a syngas to oxygenated organic compound fermentation process to be commercially viable, capital and operating costs must be sufficiently low so that it is at least competitive with alternative biomass to oxygenated organic compound processes and/or hydrocarbon based sources of such products. For instance, ethanol is commercially produced from corn in facilities having nameplate capacities of over 100 million gallons per year. Accordingly, the syngas to oxygenated organic compound fermentation process must be able to take advantage of similar economies of scale. Thus, bioreactors in a commercial scale facility may require at least 20 million liters of fermentation liquid capacity.

Various types of bioreactors are used to make the contacting of the fermentation liquid, syngas and microorganisms as efficient as possible. Nevertheless, the various bioreactor designs that are known are challenging to implement. For example, stirred tank bioreactors have high capital costs, require significant energy input for gas transfer and mixing, and need plural stages to achieve high conversion of gaseous substrates. Other syngas fermentation reactor types such a bubble column reactors and air lift (jet loop) reactors are less costly to manufacture and operate, but such bioreactors typically need microbubble spargers to make small microbubbles and these use significant amounts of energy and are prone to fouling. U.S. Pat. No. 8,795,995, discloses the use of injectors to supply gas feed to an anaerobic fermentation in a bioreactor to make liquid products such as ethanol.

The volumetric rate of fermentation liquid removal from the bioreactor may be driven by the build-up of suspended solids or by the concentration of chemical products or by-products in the fermentation liquid. In particular, a continuous syngas fermentation processes typically result in co-produced oxygenated organic compounds in addition to the sought, product oxygenated organic compound. The co-produced oxygenated organic compounds can be co-metabolites that are not desired or intermediate metabolites in the bio-production of the sought, product oxygenated organic compound. Also, co-produced oxygenated organic compounds can be produced by contaminating, or adventitious, microorganisms present in the aqueous fermentation broth. In some instances, these co-produced oxygenated organic compounds may be produced at rates, relative to the production rate of the sought product, that a build-up of the co-produced oxygenated organic compound is caused in the aqueous fermentation liquid. This build-up of the co-produced oxygenated organic compound is particularly untoward where the co-produced oxygenated organic compound reaches concentration levels that are inhibitory or toxic to the microorganisms used for the syngas fermentation. In some other instances, the co-produced oxygenated organic compound, when at sufficient concentrations, can adversely affect the metabolic pathways of certain microorganisms used for the bioconversion of syngas. For instance, where an alcohol is the sought, product oxygenated organic compound, with some microorganisms, the presence of certain concentrations of free carboxylic acids can induce a product distribution shift in which the microorganisms to generate a higher percentage of carboxylic acids. The exponentially increasing production of the acids leads to an increasing acidity in the fermentation broth causing an eventual loss of the microorganism being able to maintain cell membrane potential and loss of the population of microorganisms.

Thus, regardless of the cause for removing fermentation liquid from a bioreactor, removal and isolation of suspended solids from the fermentation liquid imposes one of the largest costs in commercially operating liquid bioconversion processes. This cost is tied to the capital and operating costs of the bioreactor system's liquid recovery zone for the recovery of the liquid from the fermentation liquid. In these systems, a product recovery step, typically comprising distillation, will produce an overhead stream containing product and a bottoms or liquid stream containing the suspended solids or bio-waste solids. Further suspended solids rejection takes place in several additional liquid recovery steps that clean-up the bottoms stream to remove suspended solids from the remaining liquid phase for recycling at least a portion of the recovered liquid to the bioreactor.

U.S. Patent Application Pub. No. 2016/0010123, which published on Jan. 14, 2016, describes a process for removing fermentation liquid containing an oxygenated organic product from an anaerobic bioconversion process. After recovery of the organic product, a remaining portion of the liquid broth undergoes anaerobic organic bioconversion to produce a fermentation liquid for recycle to the bioreactor.

The clean-up steps for removing suspended solids from the liquid routinely include centrifuges. Such centrifuge arrangements often use a bank of stacked disc centrifuges, each of which requires a relatively high capital and operating cost. Membranes have also been used to remove suspended solids from aqueous streams in processes for waste water and fermentation liquid treatment. U.S. Patent Application Pub. No. 2015/00337343, which was published on Nov. 26, 2015, describes a fermentation broth treatment method in which fermentation liquid is removed from a bioreactor via a bleed stream and/or a permeate stream. A product is removed from the bleed and/or the permeate stream to provide a product depleted stream from which a clarifying module removes solid material to provide a treated stream of liquid that returns to the bioreactor. Wu et. al. in the publication “The potential roles of granular activated carbon in anaerobic fluidized membrane bioreactors: effect on membrane fouling and membrane integrity” (published Aug. 11, 2014) describes use of a membrane bioreactor along with granular activated carbon to limit the build-up of solids on the surface of membrane in a bioreactor. U.S. Patent Application Pub. No. 2012/0118808, which was published on May 17, 2012, describes a fluidized membrane bioreactor in which fluidized particles contact the membrane and provide a support for the microorganisms.

Biological waste water treatment processes and drinking water treatment systems are known to use high-flux membranes with an outside inward flow path (permeate collected in the lumen) at very low transmembrane pressures to remove solids from streams. Often, simple hydrostatic head or modest pump suction is sufficient to provide the necessary driving force to generate permeate.

Use of a fluidized bed of granular activated carbon to continuously and gently scour membranes to reduce fouling are also known. One paper reported that by using this approach a UF-HF (ultrafilter high-rate) membrane did not require any cleaning for over half a year with only moderate loss of permeate flux rate. See Kim et. al.—“A new approach to control membrane fouling anaerobic fluidized membrane bioreactor” (published January 2015).

However, in other cases, testing has shown fouling of such membranes by retained solids on the surface of the membrane occurs relatively rapidly. Thus, the same phenomenon of rapid decline in flux rate has been shown in other arrangement that use membranes for biosolids management. Generally, as the solids concentrations on the retentate side of the membrane increase the propensity for fouling of the membrane surface also increases.

### BRIEF SUMMARY OF THE DISCLOSURE

The present disclosure generally relates to systems and methods for concentrating suspended solids prior to removal from a liquid stream. According to some embodiments, these systems and methods overcome many of the problems associated with known systems for separating suspended solids from a liquid. For example, the systems and processes of the present invention can permit the use of much smaller liquid recovery zones, such as centrifugal systems, which, in turn, can lead to significant cost savings in the installation and operation of fermentation systems.

According to one embodiment, the present disclosure describes processes and systems that convert a wide variety of abundant feedstocks, such as natural gas, wood, garbage, industrial gases and other carbon-containing materials into syngas that is then converted to liquid products such oxygenated organic products such as fuels and chemicals, using microorganisms in a fermentation liquid, which are more efficient and cost effective for removing suspended solids from the liquid stream recovered from a bioreactor than previously proposed systems and processes.

The present disclosure, therefore, provides robust processes for converting abundant feedstocks to liquid products in a fashion that can overcome some of the most significant operational challenges plaguing the efficient use of the anaerobic fermentations required for achieving commercial success of such methods.

It has now been discovered that placement of a properly configured solids concentration vessel, which can include at least one membrane or filter, between initial and finishing purification steps can remove waste or suspended solids efficiently. The disclosed processes and systems deliver a liquid stream containing suspended solids to a solids concentration vessel that produces a permeate stream and a retentate that comprises concentrated suspended solids therein. The liquid stream passes through the solids concentration vessel to produce a retentate containing most, if not all, of the suspended solids, which are provided to a liquid recovery zone for recovering additional liquid that can be recycled to the bioreactor. The bioreactor may also receive permeate liquid from the solids concentration vessel.

Advantageously, the suspended solids removal capability of a solids concentration vessel depends on the overall volume of the liquid stream provided to the solids concentration vessel and not on the concentration of suspended solids in the liquid stream provided to the solids concentration vessel. A two to four-fold increase in the concentration of solids in the input stream has been found to permit use of a relatively small liquid recovery zone to affect a nearly complete removal of all suspended solids from the liquid stream. In one exemplary embodiment, a distillation column bottoms stream with about 2 g/L was increased to a concentration of 4 to 8 g/L, which allowed recovery and/or recycle of an effluent or liquid that is essentially 100% free from suspended solids. The 4 to 8-fold increase in concentration of the distillation column bottoms stream means that the mass or volume flow rate of the input stream to the liquid recovery zone, in this case a centrifuge, is reduced to a half or a quarter, which in turn allows a reduction in the centrifugal processing capacity, and thus the cost and system complexity associated therewith, by a concomitant or proportional amount.

In one exemplary aspect, the disclosure describes a bioreactor system for producing a liquid product from syngas in a fermentation process. The system includes a bioreactor vessel adapted to contact microorganisms with a feed gas and with a liquid containing microorganisms, nutrients, adjuvants, additives and/or other solid material to produce the liquid product. Wherein said bioreactor vessel defines a bioreactor outlet for removing a liquid stream containing suspended solids and the liquid product. A product separation vessel communicates with the bioreactor outlet and is arranged to receive at least a portion of the bioreactor effluent or liquid stream. The product separation vessel can have an internal configuration arranged to produce a product stream containing the liquid product and a liquid stream containing biosolids or suspended solids at a higher concentration that exceeds that of the product stream. The product separation vessel defines a product outlet and liquid stream outlet. The solids concentration vessel retains at least one membrane or filter and the membrane or filter can be arranged in a module assembly. The at least one membrane or filter or assembly thereof communicates with the liquid stream outlet to receive the liquid stream. The membrane, filter, or module thereof is arranged to contact the liquid stream with an inlet surface of a membrane or filter and the membrane or filter preferentially permeates liquid through the inlet surface and out the opposite surface to produce a liquid permeate having a reduced concentration of suspended solids relative to the liquid stream. The inlet surface inhibits the movement of suspended solids thru the inlet surface to produce a retentate with a higher concentration of suspended solids relative to the liquid permeate. The solids concentration vessel defines a liquid permeate outlet for withdrawing the liquid permeate and a retentate outlet for withdrawing the retentate. A liquid recovery zone communicates with the retentate outlet for receiving at least a portion of the retentate and contains internals suitable for separating the retentate into a clarified stream comprising liquid and a concentrate stream comprising suspended solids at a higher concentration of suspended solids than the permeate.

In another aspect of the disclosure, the liquid recovery zone defines a clarified liquid outlet and the bioreactor is in communication with the liquid outlet to receive at least some of the clarified stream and the permeate outlet is in communication with the bioreactor vessel to receive at least a portion of the permeate stream.

In another aspect of the disclosure, the clarified stream and permeate streams are combined before being recycled. The combined streams can be recycled to a fermentor or bioreactor.

In another aspect of the disclosure, the product separator comprises a distillation column with separation trays to produce the product stream as an overhead stream and the biosolids effluent as a bottoms stream.

In another aspect of the disclosure, the solids concentration vessel retains a scouring medium adapted to contact the inlet face and move across the inlet face. The scouring medium may comprise gas, a liquid or particulate material. The solids concentration vessel may have a fluidization gas inlet for a gaseous fluidization medium and it may move the gas at an upward superficial velocity rate that keeps the fluidization medium in an agitated state. In case of particulate material, it may be granulated activated carbon, silica, aluminosilicate, ceramic, teflon or plastic particulates and has the property of being readily separated from the retentate stream within the solids concentration vessel.

In another aspect of the disclosure, the liquid recovery zone comprises at least one centrifuge.

In another aspect of the disclosure, the liquid recovery zone comprises a sedimentation separation vessel adapted to separate suspended solids using gravity.

In one embodiment, the bioreactor system is adapted to produce a liquid product from syngas that has a bioreactor vessel adapted to contact microorganisms with syngas in liquid containing microorganisms and other solid material. The bioreactor vessel defines a bioreactor outlet for removing a bioreactor effluent. A product separation vessel, which can be a distillation column, communicates with the bioreactor outlet and defines a product outlet and a liquid stream outlet and is arranged to receive at least a portion of the liquid stream. The distillation column has separation internals comprising distillation trays arranged to separate the bioreactor effluent and produce a product stream comprising the liquid product and a liquid stream containing biosolids at a higher concentration than the product stream, wherein said distillation column defines a product outlet and a liquid stream outlet.

A solids concentration vessel communicates with the liquid stream outlet to receive the liquid stream and contains a membrane or filter arranged to contact the liquid stream with an inlet surface of a membrane or filter to preferentially permeate liquid through the inlet surface and out of an outlet surface on the opposite side of the membrane or filter. The inlet surface inhibits the movement of suspended solids through the inlet surface to produce a retentate having an increased concentration of suspended solids than the liquid stream. The solids concentration vessel defines a permeate outlet for withdrawing the permeate and a retentate outlet for withdrawing the retentate. The solids concentration vessel can be adapted to retain a scouring medium that reduces build-up of suspended solids on the inlet face. The scouring media, which may be employed continuously or more preferably intermittently, allows maintenance of a high permeate flux rate. A centrifuge is arranged to communicate with the retentate outlet to receive at least a portion of the retentate and to separate the retentate into a clarified stream comprising liquid and a concentrate stream comprising biosolids or bio-waste solids and having a higher concentration of biosolids or bio-waste solids than the retentate.

In another aspect, this disclosure provides a process for producing a liquid product from syngas that passes the feed gas to a bioreactor and contacts the feed gas with microorganisms in a fermentation liquid that contains the microorganisms and produces a liquid product and biosolids. The fermentation liquid passes to a product separation vessel that separates the fermentation liquid into a product stream of liquid product and a liquid stream containing suspended solids at a higher concentration than the product stream that passes to an inlet surface of a membrane. The membrane permeates liquid from the liquid stream and excludes at least a portion of the suspended solids from passing through the membrane to produce a permeate stream having a lower concentration of suspended solids than the liquid stream and a retentate stream having a higher concentration of suspended solids than the liquid stream. The retentate passes to a liquid recovery zone and separates the retentate into a a clarified stream comprising liquid having a lower concentration of the suspended particles than the retentate and a concentrate stream having a higher concentration of suspended solids biosolids than the retentate. A portion of the permeate stream and/or the clarified stream can be returned to the bioreactor.

In another aspect, the product separator is a distillation column and that provides the product stream as an overhead stream and the liquid stream as a bottoms stream.

In another process aspect of this disclosure a gas, liquid, or particulate scouring medium contacts the inlet surface of the membrane and moves across the inlet face to remove biosolids from the inlet face. If the scouring medium is particulate material it may be granulated activated carbon, silica, aluminosilicate, ceramic and plastic particulates and the particulate material has the property of being readily separated from the retentate stream within the solids concentration vessel.

In another process aspect of the disclosure at least one centrifuge separates solids from the retentate stream.

In another process aspect, the membrane is a polymeric membrane maintained in a range of 20 to 40° C., a ceramic membrane maintained in a range of 640 to 120° C. or metallic membrane.

In another aspect, the feed gas is carbon monoxide and/or a mixture of a carbon dioxide and hydrogen and the liquid product of a C1 to C6 alkoxy compound, and preferably ethanol or butanol.

In another process aspect of the disclosure a feed gas contacts microorganisms contained in fermentation liquid to produce a liquid product and the fermentation liquid containing suspended solids passes to a distillation column to produce an overhead product stream comprising the liquid product and a biosolids effluent stream containing suspended solids. The biosolids effluent stream passes to an inlet surface of a membrane that permeates liquid from the suspended solids and excludes at least a portion of the suspended solids from passing through the membrane to produce a permeate stream having a lower concentration of biosolids than the effluent stream and a retentate stream having a higher concentration of biosolids than the effluent stream. A fluid agitates particulate matter that scours the inlet surface by passing over it. The retentate passes to a centrifuge that separates suspended solids from the retentate stream to produce a clarified stream having a lower concentration of the biosolids than the permeate stream and a concentrate stream having a higher concentration of biosolids than the permeate stream. At least a portion of the permeate stream and/or the clarified stream is recycled to the bioreactor.

### BRIEF DESCRIPTION OF THE DRAWING(S)

FIG. 1 is a schematic depiction of an apparatus that can be used in the practice of a process in accordance with the disclosure.

FIG. 2 is a schematic depiction of an apparatus that can be used in the practice of a process in accordance with the disclosure.

### DETAILED DESCRIPTION

The present disclosure relates to systems and processes for concentrating suspended solids. More specifically, the present disclosure relates to separation systems that may be used to separate suspended solids from a liquid. The present disclosure has applicability to systems and processes used to separate suspended solids, such as bio-waste solids or biosolids, from a liquid stream, such as from fermentation liquid from a bioreactor. For example, one application in which the subject matter of the present disclosure may be used is the conversion of carbon monoxide and of hydrogen and carbon dioxide to oxygenated organic compounds and, more particularly, removing suspended solids from product bioreactor effluent and providing recycle liquid that can be sent back to the bioreactor. Aspects of the present disclosure are described with respect to this exemplary application. However, it should be appreciated that the present disclosure is not limited to use in these applications. Rather, the present disclosure may have applicability to any application or system in which it may be desirable to concentrate suspended solids in a liquid.

All patents, published patent applications, unpublished patent applications and articles referenced herein are hereby incorporated by reference in their entirety. Before describing a particular embodiment for a process and system in accordance with the disclosure, it would be useful to define certain terms as used herein. The following terms have the meanings set forth below unless otherwise stated or clear from the context of their use. Use of the terms “a” and “an” is intended to include one or more of the element described.

Accordingly, the term oxygenated organic compound means one or more organic compounds containing two to six carbon atoms selected from the group of aliphatic carboxylic acids and salts, alkanols and alkoxide salts, and aldehydes. Often, oxygenated organic compound is a mixture of organic compounds produced by the microorganisms contained in the aqueous broth. The oxygenated organic compounds produced by the processes described in the present disclosure will depend upon the microorganism or combination of microorganisms used for the fermentation and the conditions of the fermentation.

The term bioreactor refers to a single vessel or an assembly of vessels suitable to contain fermentation liquid and microorganisms for the bioconversion. A bioreactor assembly may comprise one or more bioreactors which may be, with respect to gas flow, in parallel or in series flow. Each bioreactor may be of any suitable design. Bioreactors include, but are not limited to, bubble column reactors, deep tank reactors, jet loop reactors, stirred tank reactors, trickle bed reactors, and biofilm reactors including, but not limited to, membrane bioreactors and static mixer reactors including pipe reactors. Bioreactors can contain associated equipment such as injectors, recycle loops, agitators, and the like.

The terms suspended solids and/or bio-waste solids and/or biosolids and/or organic waste material means solid material composed mainly of microorganisms, microorganism residue, precipitated proteins and other particulate organic by-products.

The terms fermentation liquid and/or fermentation effluent and or bioreactor effluent means a liquid phase that retains microorganisms, feed substrate and fermentation products, which may be contained in one or more bioreactors.

The term liquid stream means a liquid phase that comprises suspended solids.

The term solid concentration vessel refers to a single vessel or an assembly of vessels suitable to concentrate suspended solids in a liquid stream. A solid concentration vessel may concentrate suspend solids by gravity, sedimentation method, comprise one or more centrifuges, or comprise a combination thereof.

The term substrate is any substance that can be maintained in the fermentation liquid and serve as a feed to microorganisms. In the case of producing oxygenated organic compounds, the substrate is a feed gas having one or more of (i) carbon monoxide and (ii) carbon dioxide and hydrogen. A feed gas substrate and may contain other components including, but not limited to, recycled off-gas or a fraction thereof and other additives, inert elements or compounds such as methane and nitrogen, and other components that can be contained in a syngas.

The term syngas means a gas, regardless of source, containing at least one of hydrogen and carbon monoxide and may, and usually does, contain carbon dioxide. Syngas is typically produced by a gasifier, reformer (steam, auto thermal or partial oxidation) and will typically contain from 10 to 60 mole % CO, from 10 to 25 mole % CO₂ and from 10 to 75, often at least about 30, and preferably between about 35 and 65, mole % H₂. The syngas may be obtained directly from gasification or from petroleum and petrochemical processing or industrial processes or may be obtained by blending two or more gas streams. Also, the syngas may be treated to remove or alter the composition including, but not limited to, removing components by chemical or physical sorption, membrane separation, and selective reaction.

Turning now to the present disclosure, the processes and methods described herein may be applied to the use of any microorganism that is suitable for the desired conversion and that will produce bio-waste in a bioreactor. A wide variety of such processes may be known or hereafter become known.

This present disclosure is useful to the bioconversion of CO and/or H2/CO2 to acetic acid, n-butanol, butyric acid, ethanol and other products. This bioconversion along with the microorganisms, substrates and products associated therewith are well known. For example, a concise description of biochemical pathways and energetics of such bioconversions have been summarized by Das, A. and L. G. Ljungdahl, Electron Transport System in Acetogens and by Drake, H. L. and K. Kusel, Diverse Physiologic Potential of Acetogens, appearing respectively as Chapters 14 and 13 of Biochemistry and Physiology of Anaerobic Bacteria, L. G. Ljungdahl eds., Springer (2003). Any suitable microorganisms that have the ability to convert the syngas components: CO, H2/CO2 individually or in combination with each other or with other components that are typically present in syngas may be utilized. Suitable microorganisms and/or growth conditions may include those disclosed in U.S. Patent Application Pub. No. 2007/0275447, entitled “Indirect Or Direct Fermentation of Biomass to Fuel Alcohol,” which discloses a biologically pure culture of the microorganism Clostridium carboxidivorans having all of the identifying characteristics of ATCC no. BAA-624; U.S. Pat. No. 7,704,723 entitled “Isolation and Characterization of Novel Clostridial Species,” which discloses a biologically pure culture of the microorganism Clostridium ragsdalei having all of the identifying characteristics of ATCC No. BAA-622; both of which are incorporated herein by reference in their entirety. Clostridium carboxidivorans may be used, for example, to ferment syngas to ethanol and/or n-butanol. Clostridium ragsdalei may be used, for example, to ferment syngas to ethanol.

Suitable microorganisms and growth conditions for converting CO and/or H2/CO2 to C4 hydrocarbons include the anaerobic bacteria Butyribacterium methylotrophicum, having the identifying characteristics of ATCC 33266 which can be adapted to CO and used and this will enable the production of n-butanol as well as butyric acid as taught in the references: “Evidence for Production of n-Butanol from Carbon Monoxide by Butyribacterium methylotrophicum,” Journal of Fermentation and Bioengineering, vol. 72, 1991, p. 58-60; “Production of butanol and ethanol from synthesis gas via fermentation,” FUEL, vol. 70, May 1991, p. 615-619. Other suitable microorganisms include: Clostridium Ljungdahlii, with strains having the identifying characteristics of ATCC 49587 (U.S. Pat. No. 5,173,429) and ATCC 55988 and 55989 (U.S. Pat. No. 6,136,577) that will enable the production of ethanol as well as acetic acid; Clostridium autoethanogemum sp. nov., an anaerobic bacterium that produces ethanol from carbon monoxide. Jamal Abrini, Henry Naveau, Edomond-Jacques Nyns, Arch Microbiol., 1994, 345-351; Archives of Microbiology 1994, 161: 345-351; and Clostridium Coskatii having the identifying characteristics of ATCC No. PTA-10522 described in U.S. Pat. No. 8,143,037.

Mixed cultures of anaerobic microorganisms useful for the bioconversions of syngas to oxygenated organic compounds as has been discussed above. The mixed cultures can be syntrophic and involve C1-fixing microorganisms and microorganisms that bioconvert the products of the C1-fixing microorganisms to higher oxygenated organic compounds. C1-fixing microorganisms include, without limitation, homoacetogens such as Clostridium ljungdahlii, Clostridium autoethanogenum, Clostridium ragsdalei, and Clostridium coskatii. Additional C1-fixing microorganisms include Alkalibaculum bacchi, Clostridium thermoaceticum, and Clostridium aceticum.

In one embodiment, it is contemplated that the aqueous fermentation broth comprises an aqueous suspension of microorganisms and various media supplements. Suitable microorganisms for CO and/or H2/CO2 generally live and grow under anaerobic conditions, meaning that dissolved oxygen is essentially absent from the fermentation broth. The various media supplements include adjuvants to the aqueous fermentation broth that may comprise buffering agents, trace metals, vitamins, salts etc. Adjustments in the fermentation broth may induce different conditions at different times such as growth and non-growth conditions which will affect the productivity of the microorganisms.

One example of an aqueous fermentation broth for bioconversion can be found in U.S. Pat. No. 7,704,723, which discloses conditions and contents of suitable aqueous fermentation broths for bioconversion CO and H2/CO2 using anaerobic microorganisms. Anaerobic fermentations of hydrogen and carbon monoxide involve the contact of a gaseous substrate-containing feed with an aqueous fermentation broth containing microorganisms capable of generating oxygenated organic compounds such as ethanol, acetic acid, propanol and n-butanol. The bioconversion of carbon monoxide results in the production of oxygenated organic compound and carbon dioxide. The conversion of hydrogen involves the consumption of hydrogen and carbon dioxide, and this conversion is sometimes referred to as the H2/CO2 conversion or, as used herein, the hydrogen conversion.

Relative to the present disclosure, a first separation of gases from the fermentation liquid takes place in the bioreactor. Unconverted portions of the gaseous feed to the bioreactor will collect in a head space of the bioreactor along with any by-product gases and vapors. Head space gas normally undergoes separation/treatment for recovery and use as an energy source and/or recycle of the tail gas back to fermentation and the possibly recovery of other valuable gas components in the tail gas. A portion of the bioreactor effluent is withdrawn from the bioreactor via a bioreactor outlet nozzle provided most commonly at the top of the bioreactor vessel. Bioreactor arrangements often recirculate a portion of the bioreactor effluent to provide mixing of the fermentation liquid in the bioreactor and as a feed injection medium to distribute feed over the entire bioreactor vessel.

At least a portion of the bioreactor effluent passes to a product separation vessel that recovers a product. The separation section may have one or more distillation columns, with each column providing an arrangement of trayed sections to recover an overhead product stream from a bottoms stream. The bottoms stream comprises a biosolids effluent that contains the suspended solids or bio-waste solids in fermentation liquid or liquid stream.

The biosolids effluent stream goes to one or more solids concentration vessels that contains at least one membrane, where the membrane can be in arrangement with other membranes. The system and process can use any membrane arrangement and membrane material that is suitable for the conditions of the separation and will provide the needed separation of the suspended solids from the liquid stream. The selection of the membrane arrangement may depend on the type of scouring material employed in the solids concentration vessel. In general, the membrane can be any type of filter media that permits at least some liquid to pass through while retaining solids on an upstream side thereof. The most typical membrane arrangements of hollow fiber, flat plate and spiral membranes may all work. Of these three, the limited access to the space between membrane faces may make spiral membranes the least suitable for scouring medium, especially for a particulate scouring material. Similarly, in the case of filters used as membranes, any type of filter media can be used and selected to retain particles of a certain size for any particular application as desired.

Membrane material types can be polymeric, ceramic, Teflon(R) or metallic membranes. Temperature conditions in the solids concentration vessel will significantly influence the choice of membrane materials. Ceramic membranes will generally accommodate a temperature range of 40 to 120° C. Whereas polymeric based membranes work best at a lower temperature range of 20 to 40° C. Preferably the solids concentration vessel operates at as high a temperature as possible to take advantage of the higher flux rates associated with higher temperatures. It may be that the use of membranes made from different materials such as ceramic or Teflon, could be cost effective based on the higher fluxes that can be maintained and resultant reduction in membrane surface area required.

Heat inputs to the distillation stage may raise the temperature of the distillation bottoms to the point that it or the solids concentration vessel may require cooling to permit the use of some membrane materials. If polymeric membranes are used some cooling to keep within the acceptable temperature range will usually be needed.

An inlet face of the membrane, or an upstream side of a filter, receives the biosolids effluent and permeates the fermentation liquid there through to the exclusion of the suspended solids. Preferential retention of the suspended solids via the membrane or filter concentrates the suspended solids into the retentate and preferably increases its concentration at least 2-fold and, typically, about 4-fold and more. From a volume flow standpoint, the concentration increase factor in the retentate is coupled by an analogous volume decrease factor of the retentate relative to the biosolids effluent.

Contact of the inlet face with the suspended solids may result in a layer or cake of these solids forming thereon. Delivering the liquid stream to the solids concentration vessel and into contact with the inlet face with sufficient turbulence may sufficiently scour the inlet face to keep it relatively free of accumulated suspended solids. However, in many cases keeping the inlet surface relatively clean will require the use of a scouring material.

Suitable scouring materials can comprise gases, liquids, solids or combinations thereof. Agitation of the scouring material as it contacts the face will increase its effectiveness. While gas and liquid material may provide sufficient scouring, in some cases, the most effective scouring comes from particulate matter.

Any suitable particulate material can be used as a scouring material or medium. Suitable scouring mediums are those that: remain in a stable form under the conditions in the solids concentration vessel and while in contact with the membrane inlet surface and the liquid stream; have the property of being readily separated from the retentate stream within the solids concentration vessel; and will not damage the membrane when it contacts the inlet face. For example, plastic media with a specific gravity less than 1.0 can be fluidized via downward liquid flow or combination of flow and gas addition to scour the surface. Upon cessation of the fluidizing flows the media rises due to the density difference to form a floating layer at the top of the solids concentration vessel.

Specific types of particulate material that may be suitable for particular applications include granulated activated carbon, silica, aluminosilicate, ceramic, Teflon(R) and plastic particulates.

Gentle scouring of the inlet face of the membrane will in most cases prevent any increase in the transverse membrane pressure. Keeping the particulate matter in a fluidized state can provide this gentle scouring. A gas or liquid stream may keep the particulate matter in a fluidized state. The liquid medium may comprise the biosolids effluent itself or an added fluidization stream. Effective particulate scouring will also keep the particulate material free of organics that may agglomerate, such as the precipitated proteins, that can form a layer of organic material on top of fluidized particulate material media in the fluidized bed. Note this scouring can be done continuously or intermittently as needed.

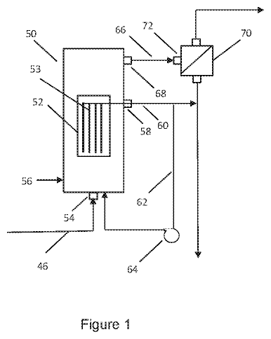

In one embodiment, the membrane inlet surfaces are immersed or submerged in the bed of fluidized particulates and particulates are fluidized by passing the biosolids effluent through the particulate. The fluidizing medium preferably passes through the particulates at a rate sufficient to completely support the buoyant weight of the particulates. To achieve and maintain sufficient fluidization, some recycle of the biosolids effluent may be required to maintain the proper flow rate as is shown in the system of FIG. 1. During operation, permeate that is free of solids is recovered from the solids concentration vessel. The permeate may be sent back to the bioreactor.

The retentate from the solids concentration vessel contains a concentrated suspended solids stream. It is still important to recover as much of the remaining liquid as possible from this stream, so the retentate is passed to another stage of separation, referred to as the liquid recovery separation that takes place in a liquid recovery zone. Any form of separator suitable for extracting liquid from a high concentration of solids may be used.

In the illustrated embodiment, the liquid recovery zone will use centrifuges. The advantage of such arrangement is that the solids concentration vessel allows a significant reduction in the number of stacked centrifuges required for the liquid recovery from the biosolids effluent. For example, if recovery of a clarified liquid stream from the biosolids effluent normally requires a bank of 4 similarly sized centrifuges, a 4-fold increase in the concentration of the solids in the stream undergoing centrifugation will decrease the required number centrifuges down to one. This reduction is possible because the total volumetric flowrate forwarded to the liquid recovery zone is decreased analogously to the increase in permeate being withdrawn before the retentate is forwarded to the liquid recovery zone. Stated differently, the liquid stream volumetric or mass flow rate will be about equal to the total volumetric or mass flow rate of the retentate and the permeate streams.

A specific aspect of the process and system as envisioned is shown in FIG. 1, which represents a schematic depiction of an apparatus for the system and suitable for practicing processes in accordance with the disclosure. FIG. 1 omits minor equipment such as pumps, compressors, valves, instruments, the exchangers and other devices the placement of which and the operation thereof are well known to those practiced in chemical engineering. FIG. 1 also omits ancillary unit operations.

The processes and operation of FIG. 1 will be described in the context of preconcentrating suspended solids in a liquid stream, such as biosolids, prior to sending the solids to the liquid recovery zone, which can be a centrifuge, but it should be appreciated that the process and method is generally applicable to other operations. The process is readily adaptable to processes that produce a bio-solid waste stream. The description in this particular context is not meant to limit the scope of the disclosure to the details presented in the following description.

Line 46 passes the liquid stream to the product separation vessel 50 via a nozzle 54 defined by the solids concentration vessel 50. The liquid stream from line 46 contacts the inlet surface (the permeating surface) of at least one hollow fiber membrane or filter 52, which can be contained in a bundle 53. A permeate passes through the at least one hollow fiber membrane or filter 52. A collector (not shown) collects the permeate from the individual membrane or filter elements and passes it out of the solids concentration vessel through an outlet 58 on solids concentration vessel 50 and into a line 60 that contains a permeate stream.

A scouring medium comprising particulate material (not shown) can optionally be used. The scouring medium, when used, circulates in a fluidized state across the entire inlet surface of the membranes or filters in bundle 53 in a continuous or intermittent fashion. The entering flow of the liquid stream from line 46 can provide or assist in the fluidization of the particles. If needed, a line 56 may supply additional fluidization gas or liquid. In addition, a portion of the permeate stream from line 60 may be recirculated via a line 62 at a rate controlled by a pump 64 to provide additional or alternate fluidization medium. Line 62 may also be used to recirculate permeate for the purpose of providing additional liquid flow across the surface of the membranes or filters in bundle 53. Recycle of liquid on the retentate side of the membranes or filters can also be used for this purpose

The retentate stream exits solids concentration vessel 50 via a line 66 though a nozzle 68 defined by the solids concentration vessel 50 and into a liquid recovery zone 70 via nozzle 72 located thereon. In this arrangement, the liquid recovery zone comprises a centrifuge that receives the retentate.

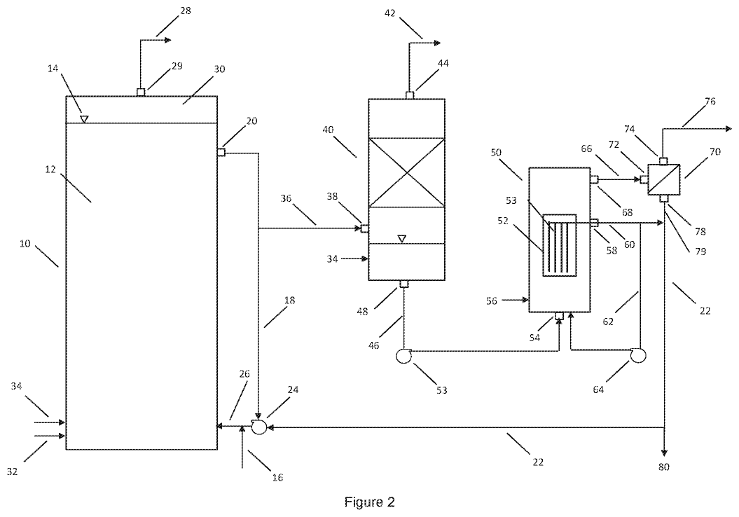

A specific aspect of the process and system as envisioned is shown in FIG. 2, which represents a schematic depiction of an apparatus for the system and suitable for practicing processes in accordance with the disclosure. FIG. 2 omits minor equipment such as pumps, compressors, valves, instruments, the exchangers and other devices the placement of which and the operation thereof are well known to those practiced in chemical engineering. FIG. 2 also omits ancillary unit operations.

The processes and operation of FIG. 2 will be described in the context of the recovery and production of ethanol, but it should be appreciated that the process and method is generally applicable to other operations. The process is readily adaptable to processes for making other oxygenated organic compounds such as well as other fermentation products that produce a bio-solid waste stream. Although shown for application in conjunction with a bioreactor in the form of a deep tank bioreactor, the processes and methods described can be used with other bioreactor designs. The bioreactor vessel keeps the microorganisms and suspended solids suspended in a fermentation liquid. The description in this particular context is not meant to limit the scope of the disclosure to the details presented in the following description.

With reference to FIG. 2, a deep tank bioreactor 10 retains a fermentation liquid 12 up to a liquid level 14. Types of bioreactors that are known to those of skill in the art have been disclosed elsewhere in this disclosure. Such bioreactors may be used alone or in combination with multiple bioreactors of the same or different types in series or parallel flow. These apparatuses will be used to develop and maintain the microorganism cultures. Preferably the bioreactor used as described in the present disclosure may provide a high conversion of carbon monoxide and hydrogen to oxygenated organic compound.

In one embodiment, the fermentation liquid is maintained under anaerobic fermentation conditions including a suitable temperature, typically between 25° C. and 60° C. and frequently in the range of about 30 to 40° C. The pH of the aqueous broth is acidic, often less than about 6.5, typically between about 4 and 6.0, and more typically between about 4.3 and 5.5.

A syngas feed 16, is combined with an injection fluid carried by line 26 that provides a motive force to disperse the feed in the form of bubbles across the bottom of the bioreactor 10. Where the sought oxygenated organic compound product is one or more alcohols, the electron to carbon ratio of the gas substrate can be between 5.5:1 to 6.5:1 and, in certain embodiments, between 5.7:1 and 6.2:1. The carbon monoxide to hydrogen mole ratio is often below about 1.1:1, and often in the range of 0.4:1 to 1:1. The rate of supply of the feed gas under steady state conditions to a fermentation bioreactor is such that the rate of transfer of carbon monoxide and hydrogen to the liquid phase matches the rate that carbon monoxide and hydrogen are converted by the microorganisms.

Injection of the feed provides mixing currents that not only assure the relatively uniform aqueous phase composition but also increase the contact time between the gas bubbles and the aqueous broth. Preferably the bubbles comprise microbubbles. The use of microbubbles promotes a stable dispersion of bubbles in the aqueous fermentation liquid. The injection fluid may comprise one or more streams from the process or an external stream. As shown in FIG. 2, a pump 24 charges a liquid recycle stream 18 and/or a recovered liquid stream from line 22 to provide injection fluid carried by line 26. The bioreactor may receive additional inputs. For example, line 34 may deliver nutrients, adjuvants and other additives to the fermentation liquid. Make-up water may be added to the fermentation liquid via line 32.

Bioreactor 10 defines a nozzle 29, through which a gas stream 28 is taken from a gas filled headspace 30 at the top of bioreactor 10. The bioreactor may be under pressure, at atmospheric pressure, or below ambient pressure. The fermentation may operate at substantially atmospheric pressure in zone 30 to reduce capital cost of the reactor.

Off gas stream 28 is essentially depleted of feed substrate but may contain a small fraction of the hydrogen and carbon oxides of the feed gas. Inert compounds or elements such as nitrogen and primarily methane will comprise a portion of the off-gas where the syngas source is steam reforming or oxygen-fed, auto thermal reforming, especially where steam or autothermal reforming of methane-containing gas is used to generate the feed gas. The depleted gas phase may also contain sulfur-containing compounds, alcohol and the like volatilized from the aqueous fermentation broth.

A portion of the off-gas may be recycled to the bioreactor (not shown). Any unrecycled off-gas may go facilities for recovery of any remaining oxygenated organic compound and remaining energy content. The ratio of recycled to exhausted off-gas can vary widely depending upon the sought conversion of syngas to oxygenated organic compound.

Any recycled off-gases may be treated to remove a portion of the carbon dioxide prior to admixture with fresh syngas. Any suitable carbon dioxide removal process may be used including amine extraction, alkaline salt extractions, water absorption, membrane or filter separation, adsorptions/desorption, and physical absorption in organic solvents.

A portion of the aqueous fermentation broth is withdrawn from line 18 via a line 36 for product recovery. For example, U.S. Pat. No. 8,211,679 shows an arrangement for a product recovery that recovers an ethanol product from a bioreactor. Product recovery includes separation and recovery of liquid products from the fermentation liquid, removal of residual cell material, return of recovered fermentation liquid and purging of waste streams and materials.

In the process and system in accordance with the disclosure, the bioreactor effluent from line 36 is provided to a product recovery zone, such as a distillation column, 40 through a nozzle 38 defined by distillation column 40. A temperature of the bioreactor effluent may be controlled by heat exchange (not shown). The distillation column 40 may function primarily as a stripping column, or may be a conventional distillation column with stripping and rectification sections. The terms stripper or stripping column and distillation column are used interchangeably herein to refer to either type of column. Preferably a step-down in pressure vaporizes at least a portion of the bioreactor effluent liquid prior to entering column 40. A pressure regulator (not shown) supplies the step-down in pressure. The liquid stream passes through an expansion valve that vaporizes all of the liquid provided via line 36.

Distillation column 40 is adapted to recover product chemicals or fuels, such as ethanol, from the withdrawn fermentation liquid. Product ethanol exits distillation column 40 via line 42 through a nozzle 44 defined by distillation column 40.

The distillation column separates the dilute bioreactor effluent stream into an overhead vapor taken as product stream 42 and a liquid stream or biosolids effluent 46 comprising ethanol depleted bottoms. The ethanol depleted bottom exits the distillation column 40 through a nozzle 48 defined by distillation column 40. Preferably the distillation column is a stripping column packed with distillation trays that are capable of handling high-solids feeds. The bioreactor effluent or liquid stream enters a stripping section of distillation column 40 (not shown). Distillation column 40 may operate under pressure, at atmospheric conditions or under vacuum. The distillation column 40 will normally provide at least 10 stages of separation.

The ethanol concentration of the bioreactor effluent in line 36 will also affect the need for any reflux of the vapor in product stream 42 or the addition of other inputs such as stream via a line 34. Typically for ethanol concentrations greater than 3 wt. % in line 42, the desired concentration of ethanol in line 44 can be attained without any recycle of the product stream directly to the column 40. For lower concentrations of ethanol in line 42, suitable condensing and reflux equipment (not shown) may be provided as necessary to achieve the desired concentrations of ethanol in product stream 42.

Line 46 withdraws the biosolids effluent or liquid stream and passes it into a solids concentration vessel 50 via a nozzle 54 defined by the solids concentration vessel 50. The biosolids effluent or liquid stream enters the solids concentration vessel 50 via line 46 through a nozzle 54 defined by the solids concentration vessel 50. A pump 53 supplies the motive force to move the stream to the shell side of solids concentration vessel 50 as needed for the at least one membrane or filter therein. The biosolids effluent or liquid stream from line 46 contacts the inlet surface (the permeating surface) of at least one hollow fiber membrane or filter contained in a bundle 53. A permeate passes through the hollow fiber membranes(s) or filter(s). A collector (not shown) collects the permeate from the individual membrane or filter elements and passes it out of the solids concentration vessel 50 through an outlet 58 on solids concentration vessel 50 and into a line 60 that contains a permeate stream.

A scouring medium comprising particulate material (not shown) can optionally be used. The scouring medium, when used, circulates in a fluidized state across the entire inlet surface of the membrane(s) or filter(s) in solids concentration vessel 50 or bundle 53 in a continuous or intermittent fashion. The entering flow of biosolids effluent from line 46 can provide or assist in the fluidization of the particles. If needed, a line 56 may supply additional fluidization gas or liquid. In addition, a portion of the permeate stream from line 60 may be recirculated via a line 62 at a rate controlled by a pump 64 to provide additional or alternate fluidization medium. Line 62 may also be used to recirculate permeate for the purpose of providing additional liquid flow across the surface of the membrane(s) or filters in solids concentration vessel 50 or in bundle 53. Recycle of liquid on the retentate side of the membrane can also be used for this purpose

The retentate stream exits the solids concentration vessel 50 via a line 66 though a nozzle 68 defined by the solids concentration vessel 50 and into a liquid recovery zone 70 via nozzle 72 located thereon. In this arrangement, the liquid recovery zone comprises a centrifuge that receives the retentate.

The centrifuge separates the concentrated solids in the retentate stream into a concentrate stream taken from a nozzle 74 by a line 76 and a clarified taken from a nozzle 78 by line 79. The concentrate stream contains essentially all of the remaining solids from the retentate stream. The clarified stream comprises mainly water, dissolved nutrients and other soluble compounds remaining in the retentate. The concentrate may be treated in any suitable manner for disposal. One such treatment is anaerobic digestion. Due to the temperatures typically used in distillation column 40, the solids are denatured.

Clarified liquid from nozzle 78 and taken by line 79 can be recycled to the bioreactor 10 via line 22. All or a portion of the permeate stream may be recycled to bioreactor vessel 10 via line 60 and line 22. A portion of the recovered water from line 60 and/or line 79 may be purged from the system via purge line 80 that withdraws liquid from line 22. This is generally done to control accumulation of dissolved solids and/or metabolites to levels where they do not inhibit the syngas fermentation.

The disclosure, in general, provides a method for reducing a number of centrifuges needed to separate solids in a retentate stream by concentrating the solids in a concentrated retentate stream and providing the concentrated stream to the centrifuge such that the centrifuge can operate at a higher efficiency. Clarified permeate can be recycled back to the bioreactor after the retentate stream has been concentrated.

In one general aspect, therefore, a process and system in accordance with the disclosure describes a system for concentrating suspended solids. The system includes a solids concentration vessel adapted for separating liquid and suspended solids, wherein a liquid stream comprising suspended solids enters the solids concentration vessel through at least one inlet. The solids concentration vessel includes at least one membrane, or filter, which is configured to contact the liquid stream and separate suspended particles in the liquid stream, from the liquid stream. The at least one membrane or filter is further configured to inhibit movement of the suspended particles through an inlet surface of the membrane or upstream side of the filter, while permitting the liquid to pass, and thus separate the liquid stream to yield: a retentate having a higher concentration of suspended particles relative to the liquid stream, and a liquid permeate stream having a lower concentration of suspended particles relative to the liquid stream. The solids concentration vessel includes a first outlet, the first outlet being fluidly in communication on a first side of the at least one membrane or filter such that the liquid permeate stream is withdrawn through the first outlet during operation, and a second outlet, the second outlet being in fluid communication on a second side of the at least one membrane or filter such that the retentate is withdrawn through the second outlet during operation. A centrifugal system is configured to separate the retentate into a clarified liquid stream and a stream comprising suspended solid particle concentrate, the stream comprising suspended solid particle concentrate being in fluid communication with the second outlet, wherein the stream comprising suspended solids concentrate has a higher concentration of solids relative to the retentate.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A system for concentrating suspended solids, the system comprising:
a solids concentration vessel adapted for separating liquid and suspended solids, wherein a liquid stream comprising suspended solids enters the solids concentration vessel through at least one inlet;
the solids concentration vessel having an internal configuration arranged to separate the suspending solids from the liquid stream comprising at least one membrane;
the at least one membrane inhibits the movement of the suspended solids through an inlet surface of the membrane to produce a retentate with an increased concentration of suspended solids relative to the suspended solids concentration of the stream;
a liquid permeate stream comprising a reduced concentration of suspended solids relative to the suspended solids concentration of the liquid stream;
the solids concentration vessel having at least two outlets, wherein the liquid permeate is withdrawn from the solids concentration vessel through a first outlet and the retentate is withdrawn through a second outlet, wherein the retentate stream of the second outlet is in communication with a liquid recovery zone to separate the retentate into a clarified liquid stream and a stream comprising suspended solids concentrate; and
wherein the stream comprising suspended solids concentrate comprises a higher concentration of solids relative to the retentate.

2. A system for concentrating suspended solids, the system comprising:
a solids concentration vessel adapted for separating liquid and suspended solids, wherein a liquid stream comprising suspended solids enters the solids concentration vessel through at least one inlet;
the solids concentration vessel including at least one filtering device, the at least one filtering device configured to contact the liquid stream and separate suspended solids in the liquid stream, from the liquid stream;
wherein the at least one filtering device is configured to inhibit movement of the suspended particles through an inlet side of the filtering device, while permitting the liquid to pass, and thus separate the liquid stream to yield:
a retentate having a higher concentration of suspended solids relative to the liquid stream, and
a liquid permeate stream having a lower concentration of suspended solids relative to the liquid stream
wherein the solids concentration vessel includes a first outlet, the first outlet being fluidly in communication with inlet side of the filtering device such that the liquid permeate stream is withdrawn through the first outlet during operation, and a second outlet, the second outlet being in fluid communication with an outlet side of the filtering device such that the retentate is withdrawn through the second outlet during operation; and
a liquid recovery zone configured to separate the retentate into a clarified liquid stream and a stream comprising suspended solid particle concentrate, the stream comprising suspended solid particle concentrate being in fluid communication with the second outlet,
wherein the stream comprising suspended solids concentrate has a higher concentration of solids relative to the retentate.

3. The system as set forth in claim 1, wherein the liquid stream comprises biosolids.

4. The system as set forth in claim 1, wherein the liquid stream comprises a liquid product.

5. The system as set forth in claim 1, wherein the liquid stream comprises a product produced by fermentation.

6. The system as set forth in claim 1, wherein the at least one filtering device is a membrane that can permeate liquid.

7. The system as set forth in claim 1, wherein the at least one filtering device is a filter.

8. The system as set forth in claim 1, wherein the liquid permeate stream has a concentration of solids that is less than 100 ppm.

9. The system as set forth in claim 1, wherein the liquid permeate stream has a concentration of solids that is less than 60 ppm.

10. The system as set forth in claim 1, wherein the liquid permeate stream has a concentration of solids that is less than 1000 ppm.

11. The system as set forth in claim 1, wherein the liquid recovery zone comprises a centrifuge device.

12. The system as set forth in claim 1, further comprising a bioreactor configured to produce the liquid stream.

13. The system as set forth in claim 1, further comprising a distillation column disposed to receive and distill a product from the liquid stream.

14. A bioreactor system for effecting a fermentation process for producing a liquid product from a gas feed stream, the bioreactor system comprising:
a bioreactor vessel adapted to contact microorganisms with a gas feed in a biosolid containing liquid that contains the microorganisms and other solid material to produce said liquid product wherein said bioreactor vessel defines a bioreactor outlet for removing a bioreactor effluent from the bioreactor vessel, the bioreactor effluent comprising fermentation liquid, biosolids and the liquid product;
a product separator vessel in fluid communication with the bioreactor outlet, the product separator vessel disposed to receive at least a portion of the bioreactor effluent, the product separator vessel comprising:
an internal configuration arranged to separate the bioreactor effluent and produce a product stream comprising the liquid product and a liquid stream containing suspended solids at a higher concentration than said product stream;
a product outlet providing the liquid product, and
a liquid stream outlet providing the liquid stream;
a solids concentration vessel having at least one membrane disposed therein, the at least one membrane being in fluid communication with the liquid stream outlet and disposed to receive the liquid stream comprising suspended solids;
wherein the solids concentration vessel is configured to separate the liquid stream into a permeate and a retentate, the retentate including a higher concentration of suspended solids than the liquid stream; and
a liquid recovery zone disposed to receive at least a portion of the retentate, the liquid recovery zone configured to separate the retentate into a clarified stream comprising liquid and a concentrate stream comprising the suspended solids and having a higher concentration of suspended solids relative to the retentate.

15. The bioreactor system of claim 14, wherein the solid concentration vessel includes a membrane having an inlet surface side and an outlet surface side, the membrane being arranged to contact the liquid stream and to allow permeate though the membrane such that a permeate comprising a liquid having a reduced concentration of suspended solids relative to the liquid stream is collected past the membrane and a retentate with an increased concentration of suspended solids relative to the liquid stream remains before the membrane.

16. The bioreactor system as set forth in claim 14, wherein the solids concentration vessel defines a permeate outlet for withdrawing the permeate and a retentate outlet for withdrawing the retentate separately from the permeate.

17. The bioreactor system as set forth in claim 14, wherein the liquid recovery zone defines a clarified liquid outlet, and wherein the bioreactor is in fluid communication with the liquid outlet to receive at least portion of the of the clarified stream.

18. The bioreactor system as set forth in claim 14, wherein the permeate outlet is in fluid communication with the bioreactor vessel and disposed to receive at least a portion of the permeate.

19. The bioreactor system as set forth in claim 14, wherein the product separation vessel comprises a distillation column operable to produce a product stream as an overhead stream and the liquid stream as a bottoms stream.

20. The bioreactor system as set forth in claim 14, further comprising a scouring medium dispersed in the solids concentration vessel, the scouring medium being circulated within the solids concentration vessel across the inlet face of the membrane.

21. The bioreactor system of claim 20, wherein the scouring medium comprises at least one of a gas, a liquid and a particulate material, and wherein the membrane includes a hollow fiber membrane.

22. The bioreactor system of claim 21, wherein the solids concentration vessel is adapted to receive a gaseous fluidization medium and maintain an upward superficial velocity of the fluidization medium at a rate that is sufficient to keep the fluidization medium in an agitated state.

23. The bioreactor system of claim 22, wherein the scouring medium comprises a particulate material selected from the group consisting of granulated activated carbon, silica, aluminosilicate, ceramic and plastic particulates, and wherein the particulate material is configured to readily separate from the retentate within the solids concentration vessel.

24. The bioreactor system of claim 23, wherein the solids concentration vessel is arranged to fluidize particles of the particulate material and wherein the solids concentration vessel includes a fluidization inlet to receive a fluidization medium.

25. The bioreactor system of claim 21, wherein the solids concentration vessel is adapted to receive a gaseous fluidization medium and maintain an upward superficial velocity of the fluidization medium at rate that will keep the fluidization medium in an agitated state.

26. The bioreactor system as set forth in claim 14, wherein the liquid recovery zone comprises a centrifuge.

27. A process for producing a liquid product from syngas comprising:
passing a feed gas to a bioreactor;
contacting the feed gas with microorganisms in a fermentation liquid that contains the microorganisms;
allowing the microorganisms to react with the feed gas and produce the liquid product and biosolids;
passing the fermentation liquid to a separator;
separating the fermentation liquid into a product stream comprising the liquid product and a biosolids effluent stream containing biosolids at a higher concentration than said product stream;
passing the biosolids effluent stream to an inlet surface of a membrane;
permeating liquid from the biosolids effluent stream and excluding at least a portion of the biosolids from passing through the membrane to produce a permeate stream having a lower concentration of biosolids than said effluent stream and a retentate stream having a higher concentration of biosolids than said effluent stream;
passing the permeate stream to a permeate separator and separating biosolids from the permeate stream to produce a clarified stream having a lower concentration of the biosolids than said permeate stream and a concentrate stream having a higher concentration of biosolids than said permeate stream; and
returning at least a portion of at least one of the permeate stream and the clarified stream to the bioreactor.

28. The process as set forth in claim 27, wherein the effluent separator comprises a distillation column and the product stream is recovered as overhead stream and the biosolids effluent is recovered as a bottoms stream.

29. The process as set forth in claim 27, wherein a scouring medium contacts the inlet surface of the membrane and moves across the inlet face to remove biosolids from the inlet face.

30. The process as set forth in claim 27, wherein the membrane comprises a hollow fiber membrane.

31. The process as set forth in claim 27, wherein the permeate separator comprises a centrifuge.

32. The process as set forth in claim 27, wherein the membrane comprises at least one of a polymeric, ceramic and metallic membrane.

33. A method for separating a biosolids effluent containing particles, from a bioreactor, the method comprising:
distilling a using a biosolids effluent to produce a product and a liquid stream;
using a filtering device to separate the liquid stream into a permeate stream and a retentate stream, the retentate stream having a higher concentration of particles than the biosolids effluent or the permeate stream; and
providing the retentate stream to a centrifuge to separate the retentate stream into a clarified fluid stream and a concentrated particle stream.

34. The method of claim 33, wherein the filtering device is one of a membrane and a filter.

35. The method of claim 33, wherein using the filtering device includes using a solids concentration vessel adapted for separating liquid and suspended solids, wherein a liquid stream comprising suspended solids enters the solids concentration vessel through at least one inlet.

36. The method as set forth in claim 33, wherein the liquid stream comprises biosolids.

37. The method as set forth in claim 33, wherein the liquid stream comprises a liquid product.

38. The method as set forth in claim 33, wherein the liquid stream comprises a product produced by fermentation.

39. The method as set forth in claim 33, wherein the filtering device is a membrane.

40. The method as set forth in claim 33, wherein the filtering device is a filter.

41. A method for separating solids suspended in a liquid stream from the liquid stream, comprising:
providing the liquid stream having a first concentration of particles suspended in the liquid stream;
filtering the liquid stream to split the liquid stream into a retentate comprising a fraction of the liquid stream and substantially all suspended solids and a permeate comprising containing a remaining fraction of the carrier fluid and almost no suspended particles; and
separating the suspended solids from the fraction of the liquid stream contained in the retentate in a liquid recovery zone.

42. The method of claim 41, wherein the filtering comprises at least one membrane.

43. The method of claim 41, wherein the liquid recovery zone comprises a centrifuge.

44. The method of claim 41 wherein the permeate and clarified liquid streams are recombined.
